(19) United States
(12) Patent Application Publication
(10) Pub. No.: US 2025/0161160 A1
(43) Pub. Date: May 22, 2025

(54) Control Device of Shallow Needling Instrument, as well as System and Method for Simulating the Features of Shallow-Needle Acupuncture

(71) Applicant: Zhongshu (Fujian) Medical Technology, Inc., Fuzhou (CN)

(72) Inventors: Jianping He, Fuzhou (CN); Feng Wang, Fuzhou (CN)

(73) Assignee: Zhongshu (Fujian) Medical Technology, Inc., Fuzhou (CN)

(21) Appl. No.: 18/944,296

(22) Filed: Nov. 12, 2024

(30) Foreign Application Priority Data
Nov. 16, 2023 (CN) 202311531392X
Jun. 12, 2024 (CN) 2024107569981

(51) Int. Cl. A61H 39/08; A61H 23/02

(52) U.S. Cl. CPC A61H 39/086; A61H 23/02; A61H 2201/5012; A61H 2201/5015; A61H 2201/5035; A61H 2201/5071

(57) ABSTRACT

The invention relates to the technical field of shallow-needle acupuncture in Traditional Chinese Medicine, and discloses a control device of a shallow acupuncture instrument, as well as a system and a method for simulating the features of the shallow-needle acupuncture techniques. The shallow acupuncture instrument includes a vibration device and a control device. The control device includes a microcontroller and a power amplifier circuit. The microcontroller is used to process the waveform based on vibration parameters acquired from external inputs, obtain the waveform data of vibration modes, and transmit the waveform data to the amplifier circuit. When the first pressure parameter acquired by the vibration device reaches the preset working threshold, the power amplifier circuit drives the vibration device to vibrate after amplifying the waveform data of vibration modes. The invention provides a control device that can drive the vibration device of the shallow acupuncture instrument to vibrate, based on the waveform data of vibration modes, generating vibration waves that can reproduce the shallow-needle acupuncture techniques, to address the difficulty in reproducing the vibration waves of shallow-needle acupuncture techniques.

### CROSS-REFERENCE TO RELATED APPLICATION

This application claims priority of Chinese application No. 202410756998.1, filed on Jun. 12, 2024, which claims priority of Chinese application No. 202311531392.X filed on Nov. 16, 2023.

### TECHNICAL FIELD

The invention relates to the technical field of shallow-needle acupuncture in Traditional Chinese Medicine, and particularly relates to a control device of a shallow acupuncture instrument, as well as a system and a method for simulating the features of the shallow-needle acupuncture techniques.

### BACKGROUND

The shallow needle therapy is to press the tip of a specially designed shallow needle on the skin surface of an acupoint or a meridian, without piercing the skin, and continuously scrap upward and downward along the handle of the needle with the middle finger nail, The shallow needle acts on a specific acupoint or meridian with a specific vibration frequency and intensity, and produces a mild and regular vibration stimulation on the acupoint or meridian to achieve Qi sensation, thereby regulating the balance of Yin and Yang in the body.

Different from the filiform-needle acupuncture, the shallow-needle acupuncture does not pierce the skin. Currently, the treatment with the shallow-needle acupuncture is overly dependent on experienced TCM practitioners. The therapeutic effects vary greatly with different practitioners. Even the same practitioner is difficult to ensure the same vibration wave is produced every time. This results in the vibration waves of the shallow-needle acupuncture being difficult to learn, replicate, and promote.

In view of the aforementioned insufficiency, it is necessary to design a control device for simulating the shallow-needle acupuncture techniques, which can reproduce the vibration waves of the shallow-needle acupuncture.

### SUMMARY

The invention provides a control device of a shallow acupuncture instrument, as well as a system and a method for simulating the features of shallow-needle acupuncture techniques, to address the difficulty in reproducing vibration waves of shallow-needle acupuncture techniques.

In the first aspect, the invention provides a control device of a shallow acupuncture instrument, wherein the shallow acupuncture instrument further includes a vibration device, and the control device includes a microcontroller and a power amplifier circuit, where the microcontroller is connected with the power amplifier circuit;

The microcontroller is used to process waveforms based on vibration parameters acquired from external inputs, obtain the waveform data of vibration modes, and transmit the obtained waveform data to the power amplifier circuit; the waveform data of vibration modes include: the original vibration time-domain waveforms of shallow-needle acupuncture techniques, and the synthesized vibration time-domain waveforms of simulating shallow-needle acupuncture techniques;

When the first pressure parameter acquired by the vibration device reaches the preset working threshold of the vibration device, the power amplifier circuit drives the vibration device to vibrate after amplifying the waveform data of vibration modes.

In the control device of the shallow acupuncture instrument provided by the invention, the microcontroller is used to process the waveforms based on vibration parameters acquired from external inputs, obtain the waveform data of vibration modes, and transmit the obtained waveform data to the amplifier circuit; the waveform data of vibration modes include: the original vibration time-domain waveforms of shallow-needle acupuncture techniques, and the synthesized vibration time-domain waveforms of simulating shallow-needle acupuncture techniques; when the first pressure parameter acquired by the vibration device reaches the preset working threshold of the vibration device, the power amplifier circuit drives the vibration device to vibrate after amplifying the waveform data of vibration modes. The invention provides a control device that can drive the vibration device of the shallow acupuncture instrument to vibrate, based on the waveform data of vibration modes, generating vibration waves that can reproduce the shallow-needle acupuncture techniques, to address the difficulty in reproducing vibration waves of shallow-needle acupuncture techniques. In an optional embodiment, the control device of the shallow acupuncture instrument further includes a pressure detection circuit that is connected to the microcontroller; the pressure detection circuit is used for calculating the pressure value of the first pressure parameter acquired by the vibration device and transmitting the pressure value to the microcontroller; and the microcontroller controls the startup and shutdown of the vibration device according to the relationship between the pressure value and the preset working threshold value of the vibration device.

In the control device of the shallow acupuncture instrument provided by the invention, the pressure detection circuit is used for calculating the pressure value of the first pressure parameter acquired by the vibration device and transmitting the pressure value to the microcontroller of the shallow acupuncture instrument; and the microcontroller controls the startup and shutdown of the vibration device according to the relationship between the pressure value and the preset working threshold value of the vibration device, thereby ensuring the effect of shallow acupuncture operation. In an optional embodiment, the control device according to claim 1, wherein both the original vibration time-domain waveforms of shallow-needle acupuncture techniques and the synthesized vibration time-domain waveforms of simulating shallow-needle acupuncture techniques include the reinforcing acupuncture waveform data, the reducing acupuncture waveform data, and the even reinforcing-reducing acupuncture waveform data which correspond to the waveforms of the reinforcing acupuncture method, the reducing acupuncture method, and the even reinforcing-reducing acupuncture method, respectively.

In the control device of the shallow acupuncture instrument provided by the invention, both the original vibration time-domain waveforms of shallow-needle acupuncture techniques and the synthesized vibration time-domain waveforms of simulating shallow-needle acupuncture techniques include the reinforcing acupuncture waveform data, the reducing acupuncture waveform data, and the even reinforcing-reducing acupuncture waveform data, which correspond to the waveforms of the reinforcing acupuncture method, the reducing acupuncture method, and the even reinforcing-reducing acupuncture method, respectively. Using three methods of acupuncture techniques, enables the shallow acupuncture instrument to better simulate shallow-needle acupuncture techniques, and generate vibration waves that can reproduce shallow-needle acupuncture techniques.

In an optional embodiment, the control device of the shallow acupuncture instrument further includes a storage module, a USB interface, an indicator light and a button which are all connected to the microcontroller;

The USB interface is used to import externally inputted vibration parameters and the second pressure parameters into the storage module, and to provide a charging interface for the control device;

The storage module is used for receiving the vibration parameters and the second pressure parameters imported via the USB interface and transmitting the vibration parameters and the second pressure parameters into the microcontroller;

The indicator light is used for displaying the working status of the shallow acupuncture instrument; and

The button is used for selecting the vibration mode of the shallow acupuncture instrument.

In an optional embodiment, the control device further includes a wireless communication module which is connected to the microcontroller and used to provide wireless communication signals to the control device.

In the control device provided by the invention, the USB interface is used to import externally inputted vibration parameters and the second pressure parameters into the storage module, and to provide a charging interface for the shallow acupuncture instrument; the storage module is used for receiving the vibration parameters and the second pressure parameters imported via the USB interface and transmitting the vibration parameters and the second pressure parameters into the microcontroller; the indicator light displays the working status of the shallow acupuncture instrument; the wireless communication module provides wireless communication signals for the control device of the shallow acupuncture instrument. The storage module, the USB interface, the indicator light, the button, and the wireless communication module, provide auxiliary functions for the control device, and provide a better basis for the control device to drive the shallow acupuncture instrument to vibrate.

In the second aspect, the invention provides a shallow acupuncture instrument comprising a vibration device and a control device as described in the first aspect above or any of its corresponding embodiments; the control device of the shallow acupuncture instrument is electrically connected to the vibration device of the shallow acupuncture instrument; the control device is used to process waveforms based on vibration parameters acquired from external inputs, and obtain the waveform data of vibration modes; when the first pressure parameter acquired by the vibration device reaches the preset working threshold of the vibration device, the control device drives the vibration device to vibrate after amplifying the waveform data of vibration modes. In the shallow acupuncture instrument provided by the invention, the control device is electrically connected to the vibration device; the control device is used to process waveforms based on vibration parameters acquired from external inputs, and obtain the waveform data of vibration modes; when the first pressure parameter acquired by the vibration device reaches the preset working threshold of the vibration device, the control device drives the vibration device to vibrate after amplifying the waveform data of vibration modes. The invention provides a control device that can drive the vibration generation component to vibrate, based on the waveform data of vibration modes, generating vibration waves that can reproduce the shallow-needle acupuncture techniques, to address the difficulty in reproducing vibration waves of shallow-needle acupuncture techniques.

In an optional embodiment, the vibration device of the shallow acupuncture instrument includes a vibrating head and a vibration generation component; the first end of the vibrating head is connected to the vibration generation component, and the second end of the vibrating head is pressed against acupuncture recipient.

The vibration generation component is used to generate vibration according to the waveform data of vibration modes which is amplified by the power amplifier circuit of the control device, to drive the vibration head to vibrate.

In the shallow acupuncture instrument provided by the invention, the vibration generation component is used to generate vibration according to the waveform data of vibration modes which is amplified by the power amplifier circuit of the control device, to drive the vibrating head to vibrate. The shallow acupuncture instrument, according to the waveform data of vibration modes, generates vibration waves that can reproduce the shallow-needle acupuncture techniques, to address the difficulty in reproducing vibration waves of shallow-needle acupuncture techniques.

In an optional embodiment, the vibration device of the shallow acupuncture instrument further includes a pressure sensor which is connected to the pressure detection circuit of the control device; the pressure sensor is used for obtaining the first pressure parameter applied by the vibrating head to acupuncture recipient when the vibration device is not in operation, and for transmitting the first pressure parameter to the pressure detection circuit of the control device; the pressure detection circuit is used to calculate the pressure value of the first pressure parameter applied by the vibrating head to acupuncture recipient when the vibration device is not in operation, and transmit the pressure value to the microcontroller of the control device; and the microcontroller controls the startup and shutdown of the vibration device according to the relationship between the pressure value and the preset working threshold value of the vibration device.

In the shallow acupuncture instrument provided by the invention, the pressure sensor is used for obtaining the first pressure parameter applied by the vibrating head contacting acupuncture recipient receiving acupuncture when the vibration device is not in operation, and for transmitting the first pressure parameter to the pressure detection circuit of the control device; the pressure detection circuit is used to calculate the pressure value of the first pressure parameter applied by the vibrating head contacting acupuncture recipient when the vibration device is not in operation, and transmit the pressure value to the microcontroller of the control device; and the microcontroller controls the startup and shutdown of the vibration device according to the relationship between the pressure value and the preset working threshold value of the vibration device, thereby ensuring the effect of shallow acupuncture operation of the shallow acupuncture instrument when it is working.

In an optional embodiment, the control device and the vibration device are of integrated design.

In the shallow acupuncture instrument provided by the invention, the control device and the vibration device are of integrated design, enabling the control device to better control the working of the vibration device.

In the third aspect, the invention provides a system for simulating the features of shallow-needle acupuncture techniques; the system including an acquisition device and the shallow-needle instrument as described in the second aspect above; the acquisition device is electrically connected to the control device. The acquisition device is used for acquiring vibration parameters of external shallow-needle acupuncture techniques;

The control device is used to process waveforms based on vibration parameters, and obtain the waveform data of vibration modes;

When the first pressure parameter acquired by the vibration device reaches the preset working threshold of the vibration device, the control device drives the vibration device to vibrate after amplifying the waveform data of vibration modes.

In the system provided by the invention for simulating the features of the shallow-needle acupuncture techniques, the control device is used to process waveforms based on vibration parameters acquired from external inputs, and obtain the waveform data of vibration modes; when the first pressure parameter acquired by the vibration device reaches the preset working threshold of the vibration device, the control device drives the shallow acupuncture instrument to vibrate after amplifying the waveform data of vibration modes. The invention provides a control device that can drive the shallow acupuncture instrument to vibrate, based on the waveform data of vibration modes, generating vibration waves that can reproduce the shallow-needle acupuncture techniques, to address the difficulty in reproducing vibration waves of shallow-needle acupuncture techniques.

In an optional embodiment, the acquisition device is further used for acquiring the second pressure parameter of external shallow-needle acupuncture techniques and transmitting the second pressure parameter to the control device.

In an optional embodiment, the acquisition device includes a vibration signal acquisition module, a pressing-pressure signal acquisition module and a data processing module; both the vibration signal acquisition module and the pressing-pressure signal acquisition module are connected to data processing module; and the results of the data processing module are transmitted to the control device;

The vibration signal acquisition module is used to acquire vibration signals of external shallow-needle acupuncture techniques and transmit the vibration signals, which are three-axis acceleration vibration signals, to the data processing module;

The pressing-pressure signal acquisition module is used to acquire pressure signals of external shallow-needle acupuncture techniques and transmit the pressing-pressure signals to the data processing module;

The data processing module is used to receive vibration signals and pressing-pressure signals, analyze and process vibration signals and pressure signals, obtain the vibration parameter and the second pressure parameter respectively, and transmit the vibration parameter and the second pressure parameter to the control device; and

The control device is further used to process waveforms based on the second pressure parameter, obtain pressing-pressure waveform data, and provide a pressing-pressure threshold value for the vibration device during vibration.

In the system provided by the invention for simulating the features of shallow-needle acupuncture techniques, the vibration signal acquisition module and the pressure signal acquisition module in the acquisition device are utilized to acquire vibration signals and pressure signals of external shallow-needle acupuncture techniques, respectively; the data processing module analyzes the vibration signals and the pressure signals, obtains the vibration parameters and the second pressure parameter respectively, and transmits the obtained parameters to the control device of the shallow acupuncture instrument. The system transforms the vibration parameters and the second pressure parameter of external shallow-needle acupuncture technique into vibration parameters and pressure parameters that can be processed by the control device of the shallow acupuncture instrument, thereby providing a parameter basis for subsequently driving the vibration of the vibration device of the shallow-needle instrument.

In the fourth aspect, the invention provides a method for simulating the features of shallow-needle acupuncture techniques, which is applicable to the system for simulating the features of shallow-needle acupuncture techniques as described in the third aspect above or any of the corresponding embodiments thereof. The method includes:

Acquiring vibration parameters of external shallow-needle acupuncture techniques; Processing waveforms based on the acquired vibration parameters to obtain waveform data of vibration modes; and

Driving the vibration device to vibrate after amplifying the waveform data of vibration modes when the first pressure parameter acquired by the vibration device reaches a preset working threshold of the vibration device.

The method provided by the invention for simulating the features of the shallow-needle acupuncture techniques, which includes acquiring vibration parameters of external shallow-needle acupuncture techniques; processing waveforms based on the acquired vibration parameters to obtain waveform data of vibration modes; driving the vibration device to vibrate after amplifying the waveform data of vibration modes when the first pressure parameter acquired by the vibration device reaches the preset working threshold of the vibration device; realizes driving vibration device of the shallow acupuncture instrument to vibrate according to the waveform data of vibration modes, generating vibration waves that can reproduce the shallow-needle acupuncture techniques, to address the difficulty in reproducing vibration waves of shallow-needle acupuncture techniques.

In the fifth aspect, the invention provides a computer device, comprising a memory and a processor which are communicatively connected, wherein computer instructions are stored in the memory, and the processor executes the method of simulating the features of shallow-needle acupuncture techniques as described in the fourth aspect by executing the computer instructions.

In the sixth aspect, the invention provides a computer-readable storage medium, wherein computer instructions are stored on the computer-readable storage medium and used to enable the computer to execute the method of simulating the features of shallow-needle acupuncture techniques as described in the fourth aspect.

### BRIEF DESCRIPTION OF THE DRAWINGS

To provide a clearer illustration of specific embodiments of the invention or technical solutions in the prior art, a brief description of the drawings necessary to describe specific embodiments or the prior art will be given below. Apparently, the drawings described below are some of the embodiments of the invention, based on which other drawings can be acquired by those of ordinary skill in the art without any inventive efforts.

FIG. 1 is the schematic diagram of a control device according to one embodiment of the invention;

FIG. 2 is another schematic diagram of a control device according to one embodiment of the invention;

FIG. 3 is the schematic diagram of a system for simulating the features of a shallow-needle acupuncture technique according to one embodiment of the invention;

FIG. 4 is another schematic diagram of a system for simulating the features of a shallow-needle acupuncture techniques according to one embodiment of the invention;

FIG. 5 is the schematic diagram of the shallow needle;

FIG. 6 is the schematic diagram of a vibration signal acquisition module and a data processing module according to one embodiment of the invention;

FIG. 7 is the schematic diagram of the bonding between a vibration signal acquisition module and a shallow needle according to one embodiment of the invention;

FIG. 8 is the time-domain waveform of X-axis vibration drawn by a microcontroller based on vibration signals according to one embodiment of the invention;

FIG. 9 is the time-domain waveform of Y-axis vibration drawn by a microcontroller based on vibration signals according to one embodiment of the invention;

FIG. 10 is the time-domain waveform of Z-axis vibration drawn by a microcontroller based on vibration signals according to one embodiment of the invention;

FIG. 11 is the schematic diagram of pressing-pressure waveforms of a shallow-needle acupuncture techniques according to one embodiment of the invention;

FIG. 12 is a part of the schematic diagram of the pressing-pressure waveforms of a shallow-needle acupuncture techniques, extracted from FIG. 11 according to one embodiment of the invention;

FIG. 13 is the schematic diagram of three-dimensional structure of a shallow acupuncture instrument according to one embodiment of the invention;

FIG. 14 is the schematic diagram of three-dimensional structure of a shallow acupuncture instrument as described in FIG. 13 with the first casing removed according to one embodiment of the invention;

FIG. 15 is the schematic diagram of three-dimensional structure of a shallow acupuncture instrument from another perspective according to one embodiment of the invention;

FIG. 16 is the schematic diagram of some components of a shallow acupuncture instrument according to one embodiment of the invention;

FIG. 17 is the schematic diagram of three-dimensional structure of the first fixing structure and a vibrating head of a shallow acupuncture instrument according to one embodiment of the invention;

FIG. 18 is the partial section view of a shallow acupuncture instrument according to one embodiment of the invention;

FIG. 19 is the schematic diagram of some internal structures of a shallow acupuncture instrument according to one embodiment of the invention;

FIG. 20 is the flow diagram of a method for simulating the features of a shallow-needle acupuncture technique according to one embodiment of the invention;

FIG. 21 is the schematic diagram of a computer hardware in one embodiment of the invention.

### DETAILED DESCRIPTION OF EMBODIMENTS

To make clearer the objectives, technical solutions and advantages of the embodiments of the invention, a clear and complete description of the technical solutions in the invention will be given below, in combination with the drawings corresponding to the embodiments of the invention. Apparently, the embodiments described below are some, but not all, of the embodiments of the invention. All of the other embodiments, obtained by those of ordinary skill in the art on the basis of the embodiments of the invention without any inventive efforts, fall into/within the protection scope of the invention.

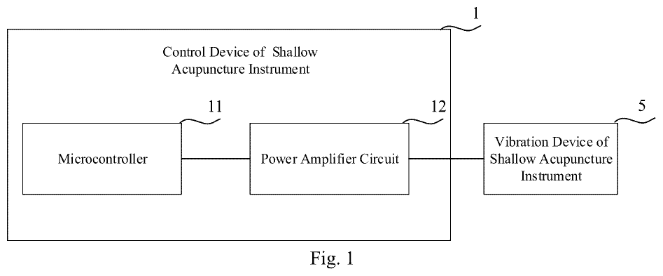

In an embodiment of the invention, a control device 1 of a shallow acupuncture instrument is provided, and FIG. 1 is the schematic diagram of the control device 1 according to the embodiment of the invention. As shown in FIG. 1, the control device is applied in a shallow acupuncture instrument which further includes a vibration device 5, and the control device 1 includes a microcontroller 11 and a power amplifier circuit 12, where the power amplifier circuit 12 is connected to the microcontroller 11. The microcontroller 11 is used to process the waveforms based on vibration parameters acquired from external inputs, obtain the waveform data of vibration modes, and transmit the obtained waveform data to the amplifier circuit; the waveform data of vibration modes include: the original vibration time-domain waveforms of shallow-needle acupuncture techniques, and the synthesized vibration time-domain waveforms of simulating shallow-needle acupuncture techniques; when the first pressure parameter acquired by the vibration device reaches the preset working threshold of the vibration device, the power amplifier circuit 12 drives the vibration device to vibrate after amplifying the waveform data of vibration modes.

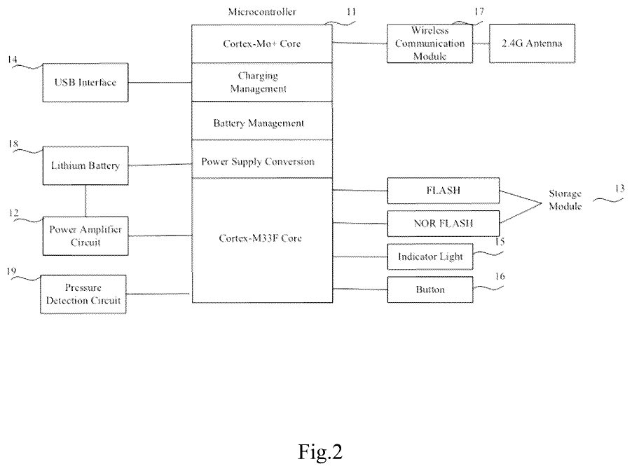

Specifically, as shown in FIG. 2, the microcontroller is implemented using a main chip DA14695 with built-in dual-core microcontroller unit (MCU). The dual cores are a Cortex-M33F core and a Cortex-M0+ core, respectively. The Cortex-M33F core is connected to the power amplifier circuit for driving and controlling the power amplifier circuit. The first pressure parameter acquired by the vibration device refers to the pressure exerted by the vibration device onto the acupuncture recipient when it is tied on the acupuncture recipient but not in operation. Presented in the form of pressure fluctuation data, the first pressure parameter is the pressure exerted by the vibrating head.

The power amplifier circuit is implemented using an amplifier chip which is an MAX98357 amplifier chip with a single power supply of 2.5V to 5.5V, for example, a 5V power supply with a load of 4Ω and a maximum output of 3.2 W. When the first pressure parameter acquired by the vibration device reaches the preset working threshold of the vibration device, the power amplifier circuit is used to amplify the waveform data of vibration modes to obtain more precise waveforms, and the amplified waveform data drives the vibration device 5 to vibrate and simulate shallow-needle acupuncture techniques to perform acupuncture on the acupuncture recipient. As shown in FIG. 2, the control device of the shallow acupuncture instrument further includes a pressure detection circuit 19 that is connected to the microcontroller; the pressure detection circuit is used for calculating the pressure value of the first pressure parameter acquired by the vibration device and transmitting the pressure value to the microcontroller; and the microcontroller controls the startup and shutdown of the vibration device according to the relationship between the pressure value and the preset working threshold value of the vibration device.

The waveform data of vibration modes may be presented with vibration time-domain waveforms. The original vibration waveform data of acupuncture techniques are presented with the original vibration time-domain waveform of acupuncture techniques, and the synthesized vibration waveform data simulating acupuncture techniques are presented with the synthesized vibration time-domain waveforms simulating acupuncture techniques, so that the vibration device 5 has two vibration modes. The original vibration time-domain waveforms of acupuncture techniques are PCM waveform sequence data that replicating the acupuncture techniques of acupuncturists.

Exemplarily, the original vibration time-domain waveform data of acupuncture techniques are the waveform data of the shallow-needle acupuncture techniques of TCM practitioners which are the time-domain waveform amplitude data obtained through sampling procedures performed at a fixed sampling rate, while the time-domain waveform amplitude data may be presented as PCM waveform sequence data. The shallow acupuncture instrument reads the waveform amplitude data, generates PCM waveform sequence data that replicate the shallow-needle acupuncture techniques of TCM practitioners, and transmits the PCM waveform sequence data to the digital power amplifier circuit which will amplify the data to drive the vibration device to vibrate, driving the vibration of the vibrating head which is in contact with the acupuncture recipient.

The synthesized vibration time-domain waveform data of simulating acupuncture techniques, is generated through the low-frequency modulation of amplitude of intermediate-frequency wave. The frequency of intermediate-frequency sinusoidal wave is about 200 Hz. The frequency of low-frequency half-sinusoidal wave is about 0.5 Hz. The configuration script data of the synthesized waveforms in the control device of the shallow acupuncture instrument, is used to generate the PCM waveform sequence data of simulating shallow-needle acupuncture techniques, which is sent to the power amplifier circuit to drive the vibration device, and driving the vibration of the vibrating head which is in contact with the acupuncture recipient.

Both the original vibration time-domain waveforms of shallow-needle acupuncture techniques and the synthesized vibration time-domain waveforms of simulating shallow-needle acupuncture techniques include the reinforcing acupuncture waveform data, the reducing acupuncture waveform data, and the even reinforcing-reducing acupuncture waveform data which correspond to the waveforms of the reinforcing acupuncture method, the reducing acupuncture method, and the even reinforcing-reducing acupuncture method, respectively.

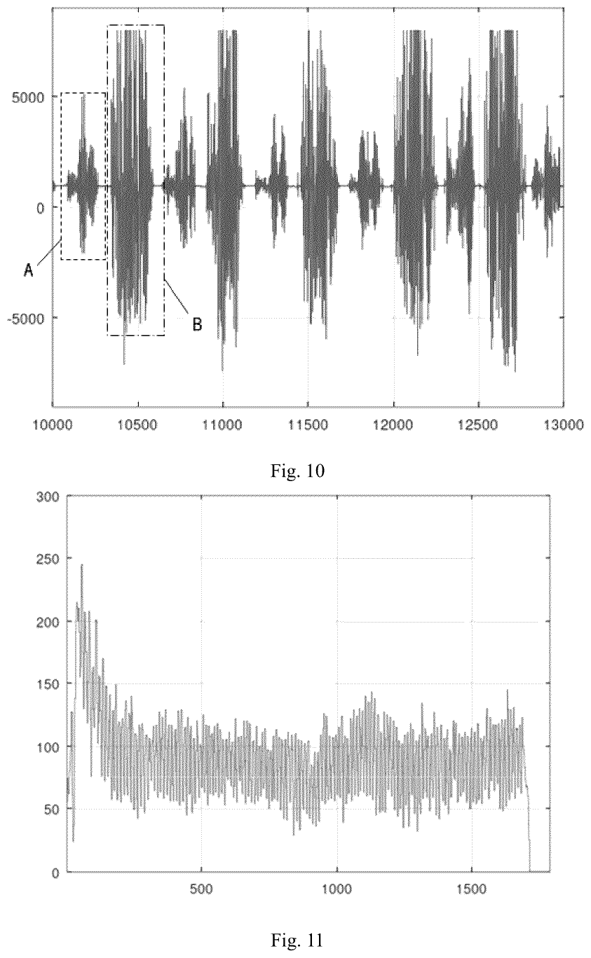

Particularly, as shown in FIG. 10, the waveform of the reinforcing acupuncture method corresponds to the acupuncture technique of lightly upward scraping and heavily downward scraping, featuring the vibration waveform amplitude of upward scraping in the first half (as Wave A in the first amplitude range shown in FIG. 10) is lower than that of the downward scraping in the second half (as Wave B in the second amplitude range shown in FIG. 10). The waveform of the reducing acupuncture method corresponds to the acupuncture technique of heavily upward scraping and lightly downward scraping, featuring the vibration waveform amplitude of upward scraping in the first half (as Wave B in the first amplitude range shown in FIG. 10) is greater than that of the downward scraping in the second half (as Wave A in the second amplitude range shown in FIG. 10). The waveform of the even reinforcing-reducing acupuncture method corresponds to the acupuncture technique of evenly upward and downward scraping, featuring the vibration waveform amplitude of upward scraping in the first half basically equals to that of downward scraping in the second half. The waveforms of the reinforcing method, the reducing method and the even reinforcing-reducing method enable the vibration device 5 to have three modes of shallow acupuncture techniques (reinforcing, reducing and even reinforcing-reducing) to choose, specifically using another set of buttons on the control device 1.

As shown in FIG. 2, the control device of the shallow acupuncture instrument further includes a storage module 13, a USB interface 14, an indicator light 15, a button 16 and a wireless communication module 17, which are all connected to the microcontroller 11.

The USB interface 14 is used to import externally inputted vibration parameters and the second pressure parameters into the storage module 13, and to provide a charging interface for the control device 1.

The storage module is used for receiving the vibration parameters and the second pressure parameters imported via the USB interface, and transmitting the vibration parameters and the second pressure parameters into the microcontroller; Specifically, the storage module is implemented using memory chips which include an 8 GB high-capacity FLASH and a 2 MB NOR FLASH, wherein the 8 GB high-capacity FLASH is used to store the waveform data of vibration modes and the pressing-pressure waveform data of shallow-needle acupuncture techniques, and the 2 MB NOR FLASH is used to store the running program of the main control chip DA14695. The vibration parameter refers to the vibration parameters of the traditional acupuncture techniques applied by the TCM practitioners to acupuncture recipients. The second pressure parameter refers to the pressure parameter of the traditional acupuncture techniques applied by the TCM practitioners to acupuncture recipients, which is the pressure at the tip of the shallow needle 4 when it is scraped.

The indicator light is used to display the working status of the vibration device; the button is used to select the vibration mode of the vibration device; the button, the indicator light and the storage module are all driven by the Cortex-M33F core of the main control chip. Particularly, the indicator light includes red, blue and green lights, and displays different states such as light on, light off, and blinking, based on the control instructions of the Cortex-M33F core.

As shown in FIG. 2, the wireless communication module is used to provide wireless communication signals for the control device. Particularly, the wireless communication module can be implemented using a 2.4G transceiver module with a low-power bluetooth function, which is driven by the Cortex-M0+ core of the main control chip. The 2.4G transceiver module is connected to an external 2.4G antenna, enabling the control device to have the transceiving function of wireless communication, and realizing the wireless communication control of the control device of the shallow acupuncture instrument by connecting to the APP software on mobile phones.

As shown in FIG. 2, the control device further includes a lithium battery 18 which is charged through the USB interface for the control device of the shallow acupuncture instrument. The main control chip DA14695 of the microcontroller further includes a battery level detection module, a charging management module, and a power supply conversion module. The lithium battery is connected to the battery level detection module, the charging management module and the power supply conversion module, which enables the charging, battery level detection and power supply control functions of the microcontroller, provides more auxiliary functions for the working of the control device of the shallow acupuncture instrument, and facilitates the operations for users.

The embodiment further provides a shallow acupuncture instrument which includes a vibration device and a control device as shown in FIGS. 1 and 2; the control device 1 is electrically connected to the vibration device 5;

The control device 1 is used to process waveforms based on vibration parameters acquired from external inputs, and obtain the waveform data of vibration modes; when the first pressure parameter acquired by the vibration device reaches a preset working threshold of the vibration device, the control device drives the vibration device 5 to vibrate after amplifying the waveform data of vibration modes.

As shown in FIG. 18, the vibration device 5 provided by the embodiment includes a vibrating head 50 and a vibration generation component 51, wherein the first end of the vibrating head 50 is connected to the vibration generation component 51, and the second end of the vibrating head 50 is contacting the acupuncture recipient (in contact with the acupoints of the acupuncture recipient). The vibration generation component 51 is used to drive the vibrating head 50 to vibrate based on the waveform data of vibration modes and the pressing-pressure data of shallow-needle acupuncture techniques.

Particularly, as shown in FIG. 13, the control device 1 and the vibration device 5 are of integrated design, and the vibration device 5 further includes a casing 5a, a limiting element 52, a limiting base 53, and a fixing structure 54 which together form a vibrator structure in the shallow acupuncture instrument. The vibrator structure and the control device 1 are provided in the casing 5a.

As shown in FIGS. 13 and 15, the casing 5a includes the first casing and the second casing that are capped together. The casing 5a is provided with a button 16, a supporting element 5c, a USB interface 14, and an indicator light 15. The supporting element 5c is in contact with the acupuncture recipient, and is below the vibrating head 50.

As shown in FIG. 18, the vibrating head 50 includes a bottom 500 and a tip 501. the bottom 500 has a dimension greater than that of the tip 501. The bottom 500 is arranged within the casing 5a and the tip 501 protrudes out of one side of the casing 5a. The vibrating head 50 is made of metallic materials.

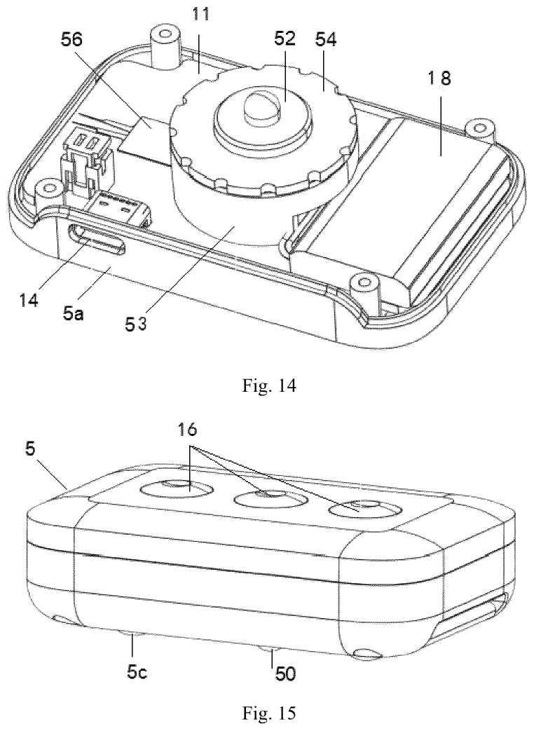

As shown in FIG. 14, the limiting base 53 is a cavity in shape, the vibration generation component 51 is arranged in the limiting base 53, and the position of the vibration generation component 51 is limited by the limiting base 53. In this embodiment, the vibration generation component 51 is a bone-conduction vibrator which, apparently, may be replaced with a flat vibrator.

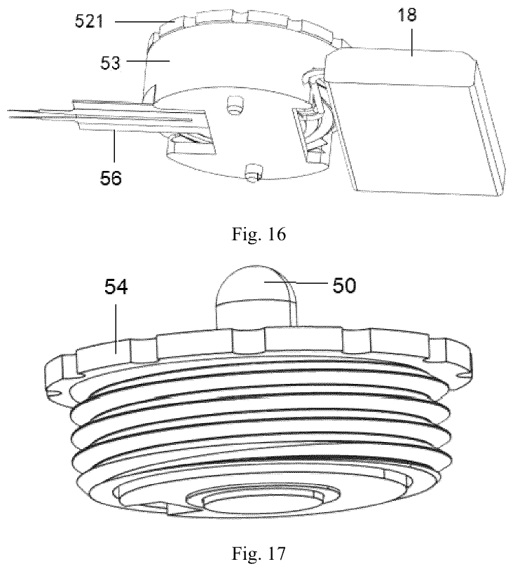

As shown in FIGS. 16, 17 and 18, the limiting element 52 is provided with a skirt 521 and a base 520. The base 520 protrudes slightly out of the casing 5a, but below the tip 501 of the vibrating head 50. the base 520 presses against the bottom 500 of the vibrating head 50 so that the vibrating head 50 can be in good contact with the vibration generation component 51. The skirt 521 is in contact with the limiting base 53. In this embodiment, the limiting element 52 is a silicone sleeve, and the tip 501 of the vibrating head 50 protrudes out of the casing 5a through a hole in the silicone sleeve. The silicone sleeve protrudes outside the casing 5a, limits the vibrating head 50 from swinging significantly, and allows the vibrating head 50 to swing to a small extent relying on the elasticity of the sleeve.

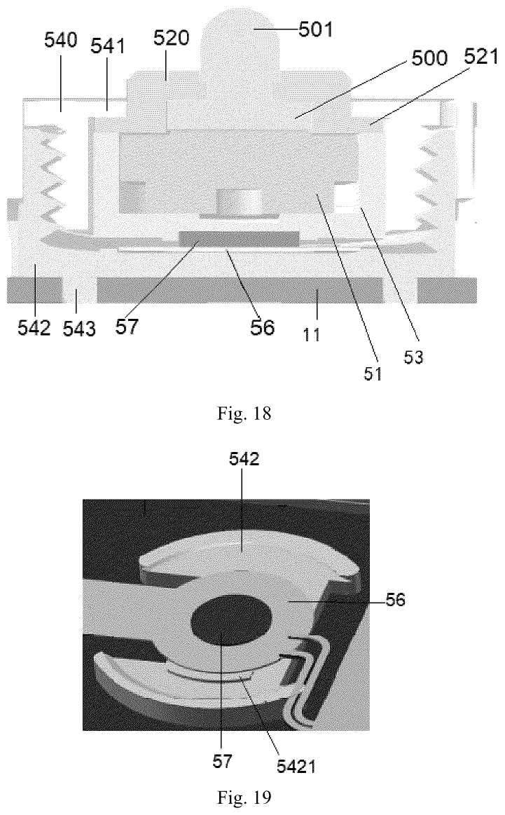

As shown in FIGS. 16, 17 and 18, the fixing structure 54 includes the first fixing structure 540 and the second fixing structure 542 which are connected together by threads. A accommodation cavity is formed within the first fixing structure 540 and the second fixing structure 542. The limiting base 53 and the vibration generation component 51 are arranged within the accommodation cavity, and the pressure monitoring element 56 is placed on the inner bottom wall of the second fixing structure 542 and is limited by the limit ribs 5421 on the second fixing structure 542. In this embodiment, as shown in FIG. 19, two limit ribs 5421 are symmetrically provided. The first fixing structure 540 is designed with a pressing frame 541, which presses the skirt 521 of the limiting element 52 towards the limiting base 53, ensuring the contact between the vibrating head 50 and the vibration generation component 51. A positioning pin 543 is provided on the second fixing structure 542.

The vibration device 5 further includes a pressure sensor which is a pressure monitoring element 56 connected to the control device 1. Before the vibration device vibrates, the pressure monitoring element is used to obtain the first pressure parameter which refers to the pressure exerted by the vibration device when it is tied onto the acupuncture recipient. The first pressure parameter is presented as pressure fluctuation data which are transmitted to the control device. The pressure detection circuit in the control device calculates the pressure value of the first pressure parameter obtained by the vibration device, and transmits the parameter to the microcontroller of the control device; and the microcontroller controls the startup and shutdown of the vibration device according to the relationship between the pressure value and the preset working threshold value of the vibration device. Particularly, the pressure monitoring element 56 is in contact with the bottom of the limiting base 53, and when the pressure monitoring element 56 is a thin-film pressure monitoring element 56, a pressure conducting member is provided between the limiting base 53 and the pressure monitoring element 56. In this embodiment, the pressure conducting member is a silicone sheet 57. When the pressure monitoring element 56 is a pressure sensing chip, the silicone sheet 57 may not be provided.

When the pressure monitoring element 56 and the pressing-pressure signal acquisition module 22 are in work, the first pressure parameter can be provided for the vibration device 5 as a reference for acupuncture. The control device judges that: if the pressure value does not reach the range of the preset working threshold of the vibration device, the vibration device 5 will not vibrate; only when the pressure value reaches the range of the preset working threshold, the vibration device 5 will vibrate, ensuring appropriate pressing-pressure on the acupuncture recipient, thereby ensuring the effect of shallow acupuncture operation.

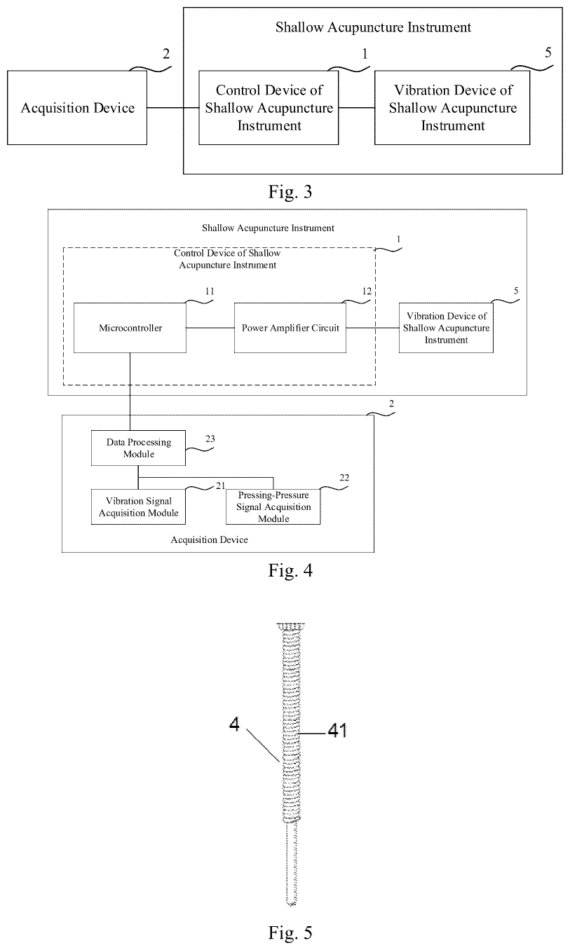

The embodiment further provides a system for simulating the features of shallow-needle acupuncture techniques. As shown in FIG. 3, the system includes an acquisition device and a shallow acupuncture instrument shown in FIGS. 13 to 19 above. The shallow acupuncture instrument includes a control device 1 and a vibration device 5 shown in FIGS. 1 and 2 above; the control device 1 is electrically connected to the acquisition device 2 and the vibration device 5, respectively;

The acquisition device 2 is used for acquiring vibration parameters and the second pressure parameter of external shallow-needle acupuncture techniques and transmitting these parameters to the control device.

As shown in FIG. 4, the acquisition device 2 includes a vibration signal acquisition module 21, a pressing-pressure signal acquisition module 22 and a data processing module 23; both the vibration signal acquisition module 21 and the pressing-pressure signal acquisition module 22 are connected to data processing module 23; and the data processing module 23 is connected to the control device.

The vibration signal acquisition module 21 is used to acquire vibration signals of external shallow-needle acupuncture techniques and transmit the vibration signals to the data processing module 23, wherein the vibration signals are acceleration vibration signals along the direction of three axes. Particularly, the acceleration vibration signals along the direction of three axes are acceleration vibration signals on the X-axis, Y-axis and Z-axis. As shown in FIG. 5, the vibration signal acquisition module 21 is used to acquire the vibration signals generated when the helix coil 41 on the shallow needle 4 is scraped (the shallow needle 4 is a manually operated acupuncture needle by itself alone). The data processing module 23 is electrically connected to the vibration signal acquisition module 21, to receive and analyze and process the vibration signals to obtain vibration parameters.

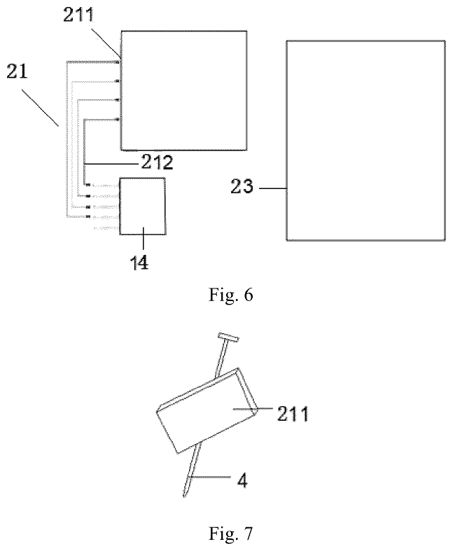

As shown in FIG. 6, the vibration signal acquisition module 21 includes an acceleration sensor 211 and a wire 212. As shown in FIG. 7, the acceleration sensor 211 is bonded to one side of the shallow needle 4 with glue so that the acceleration sensor 211 is perpendicular to the lengthwise direction of the shallow needle 4. This setup makes the vibration by scraping shallow needle 4 become most significant along the Z-axis direction of the acceleration sensor 211. One end of the wire 212 is connected to the acceleration sensor 211, and the other end is connected to the data processing module 23 via the USB interface. The data processing module 23 is a computer. The acceleration sensor 211 is a mature commercial product, and its structure will not be elaborated here.

As shown in FIG. 7, the shallow needle 4 is fixed on the side of the acceleration sensor 211 during acquisition. Scraping the helix coil 41 on the shallow needle 4 makes the shallow needle 4 vibrate. Using the acceleration sensor with supporting software to acquire the three-axe vibration acceleration values. The data processing module 23 performs data screening and data denoising on the vibration signals, obtains vibration parameters, and transmits the parameters to the control device 1.

The pressing-pressure signal acquisition module 22 is used to acquire pressure signals of external shallow-needle acupuncture techniques and transmit the pressing-pressure signals to the data processing module 23; the data processing module 23 is electrically connected to the pressing-pressure signal acquisition module 22, receives, analyzes and processes the pressing-pressure signals, and obtains the second pressure parameter. Particularly, the pressing-pressure signal acquisition module 222 includes a push-pull force gauge and a wire. The push-pull force gauge is connected to the data processing module 23 via the wire. During acquisition, the acupuncturist presses the bottom of the shallow needle 4, so that the tip of the shallow needle 4 is vertically pressed against the push-pull force gauge which acquires 1,700 pressing-pressure data for about 170 seconds, and generates 1,700 pressing-pressure signals. These pressure signals are transmitted to the data processing module 23 through the wire. The data processing module 23 performs data screening and data denoising on the pressure signals, obtains the pressure parameters and transmits the parameters to the control device 1.

The control device 1 is used to store vibration parameters and the second pressure parameters of shallow-needle acupuncture techniques, process waveforms based on the vibration parameters and the second pressure parameters, and obtain the waveform data of vibration modes and the pressing-pressure waveform data. When the first pressure parameter obtained by the vibration device reaches the preset working threshold of the vibration device, the control device drives the vibration device 5 to vibrate after amplifying the waveform data of vibration modes. The pressing-pressure waveform data are obtained to provide a reference for the pressing-pressure threshold of the vibration device for vibration.

Particularly, the vibration parameters are stored in the storage module of the control device via the USB interface, and the vibration parameters are presented in the form of vibration time-domain waveforms and vibration spectrum after waveforms are processed by the microcontroller. The generated vibration time-domain waveforms include the original vibration time-domain waveforms of acupuncture techniques and the synthesized vibration time-domain waveforms of simulating acupuncture techniques, so that the vibration device 5 has two vibration modes, which can be selected specifically by a set of buttons on the control device. The original vibration time-domain waveform of acupuncture techniques is the PCM waveform sequence data that replicates the acupuncture techniques of acupuncturist.

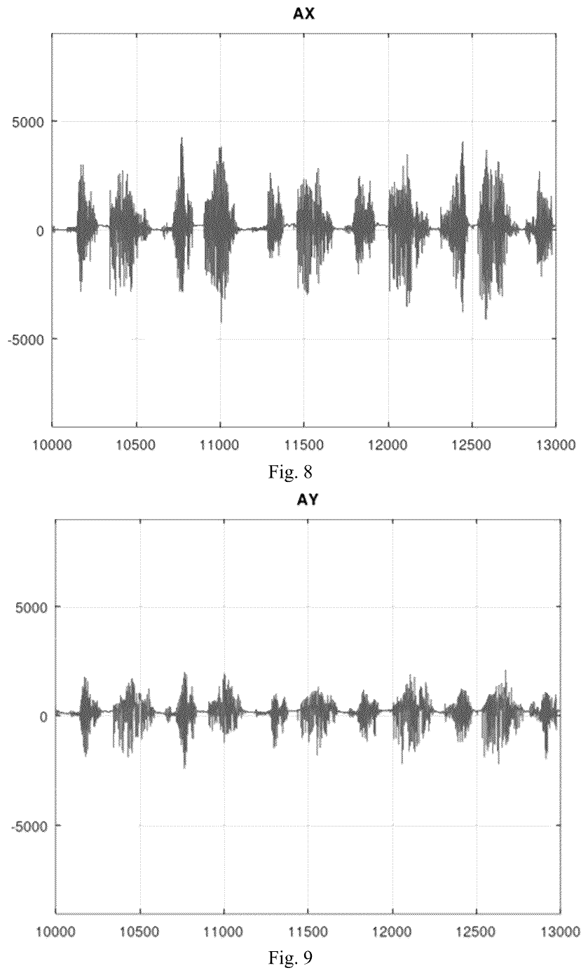

FIGS. 8, 9, and 10 are typical vibration time-domain waveforms corresponding to X, Y, and Z axes, respectively. As shown in these figures, Wave A in the first amplitude range (shown in FIG. 10) and Wave B in the second amplitude range (shown in FIG. 10) alternatively present on all the three axes (namely X, Y, and Z axes), with the most significant effect on the Z axis. The adjacent first-amplitude wave and second-amplitude wave, correspond to one complete upward and downward scraping operation of the shallow needle 4 for reinforcing or reducing method. The Z-axis is the direction perpendicular to the human body surface. The vibration along Z-axis is the major direction of upward and downward vibration generated by upward and downward scraping of shallow needle coil, and also the major physical energy that the shallow needle acts on the human body. The X-axis and Y-axis are directions parallel to the human body surface, they are additional directions of vibration generated by upward and downward scraping of shallow needle coil. The vibration along X-axis and Y-axis is not the major physical energy acting on the human body. FIGS. 8, 9, and 10, are typical vibration time-domain waveforms corresponding to X, Y, and Z axes respectively. The vibration amplitude on Z-axis is significantly greater than that on X-axis and Y-axis. The first and the second amplitude waves on Z-axis clearly correspond to the light and heavy features of shallow needle scraping techniques, while the first and second amplitude waves on X-axis and Y-axis do not clearly correspond to the light and heavy features of the shallow needle scraping techniques. Therefore, the vibration waveform implemented by the shallow acupuncture instrument select the Z-axis waveform data, and generate vibration on the Z-axis direction.

Particularly, the original vibration time-domain waveforms of acupuncture techniques and the synthesized vibration time-domain waveforms of simulating acupuncture techniques include: the waveform of the reinforcing acupuncture method, the waveform of the reducing acupuncture method, and the waveform of the even reinforcing-reducing acupuncture method. As shown in FIG. 10, the waveform of the reinforcing acupuncture method corresponds to the acupuncture technique of lightly upward scraping and heavily downward scraping, featuring the vibration waveform amplitude of upward scraping in the first half (as Wave A in the first amplitude range shown in FIG. 10) is lower than that of the downward scraping in the second half (as Wave B in the second amplitude range shown in FIG. 10). The waveform of the reducing acupuncture method corresponds to the acupuncture technique of heavily upward scraping and lightly downward scraping, featuring the vibration waveform amplitude of upward scraping in the first half (as Wave B in the first amplitude range shown in FIG. 13) is greater than that of the downward scraping in the second half (as Wave A in the second amplitude range shown in FIG. 10). The waveform of the even reinforcing-reducing acupuncture method corresponds to the acupuncture technique of evenly upward and downward scraping, featuring the vibration waveform amplitude of upward scraping in the first half basically equals to that of downward scraping in the second half.

The waveforms of the reinforcing acupuncture method, the reducing acupuncture method and the even reinforcing-reducing acupuncture method enable the vibration device 5 to have three modes of shallow acupuncture techniques (reinforcing, reducing and even reinforcing-reducing), which can be selected specifically by another set of buttons on the control device.

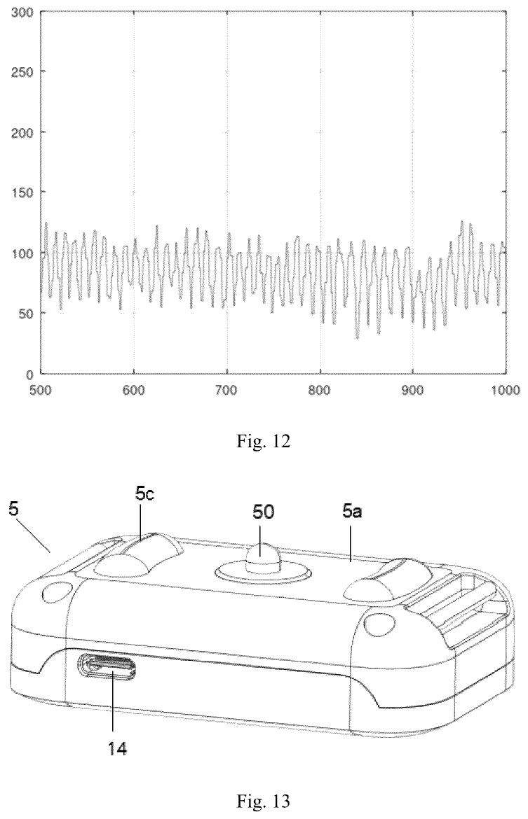

The control device obtains the pressure waveform data after performing waveform processing based on the second pressure parameter, and the pressing-pressure waveform data can provide a pressure value reference to the vibration device for vibration. Particularly, the data processing module 23 selects 500 pressing-pressure signals at a stable period (with a duration of about 50 seconds, 45 fluctuation cycles, and an average cycle of 1.1 seconds) from 1,700 pressing-pressure signals (shown in FIG. 11). The corresponding pressing-pressure waveforms of the shallow-needle acupuncture technique after waveform processing are shown in FIG. 12, and the pressing-pressure signals are used to obtain the following results: the average pressing-pressure is 84 g, with the maximum value being 126 g, and the minimum value being 29 g. Referring to four expert doctors in the rehabilitation department of a rehabilitation hospital, each of them used three shallow-needle acupuncture techniques (reinforcing, reducing and even reinforcing-reducing, respectively), a total of 12 groups of pressing-pressure data are acquired, and the pressing pressure range is determined as 53 g to 152 g, in order to avoid that the pressing pressure is too small to affect the effect of shallow acupuncture operation. Apparently, the number of pressing-pressure parameters acquired by the push-pull force gauge can also be other values, and the number of selected pressing-pressure signals at a stable period can also be other values.

It should be noted that the pressing pressure acquired by the push-pull force gauge is a pressing pressure formed by the superposition of three factors: the first factor is a relatively constant pressure generated by the acupuncturist by pressing on the bottom of the shallow needle 4; the second factor is a pressure fluctuation at about 1 Hz with the movement of the shallow needle 4; and the third factor is a vibration wave of about 200 Hz brought about by scraping the helix coil 41 on the shallow needle 4, i.e., the pressing-pressure is not constant, which means the pressure waveform data of the shallow-needle acupuncture technique includes pressure waveform data of both constant pressure and variable pressure.

The waveform data of vibration modes are obtained by the control device 1, and the power amplifier circuit 12 in the control device 1 amplifies the waveform data of vibration modes and drives the vibration device 5 to vibrate. The control device 1 also processes waveforms based on the second pressure parameter, and the obtained pressing-pressure waveform data provide a reference for the pressing-pressure threshold value to the vibration device for vibration.

The working process of the system provided in the embodiment for simulating the features of a shallow-needle acupuncture technique is described below:

The shallow acupuncture instrument is tied onto the acupuncture recipient with straps, and the vibration device 5 is turned on;

Appropriate vibration modes and acupuncture techniques are selected with the button; The pressure monitoring element monitors the first pressure parameter of the vibrating head 50 on the acupuncture recipient, i.e., the pressing pressure (presented in the form of a pressure wave), and the first pressure parameter is transmitted to the detection circuit of the control device 1. The pressure detection circuit is used for calculating the pressure value of the first pressure parameter obtained by the vibration device and transmitting the pressure value to the microcontroller which compares the pressure value with the minimum threshold value (in this embodiment, the minimum threshold value of pressing pressure is 53 g) and the maximum threshold value (in this embodiment, the maximum threshold value of pressing pressure is 152 g). If the pressure value is less than the minimum threshold value, the vibration device 5 does not enter the vibration working mode because the effect of shallow acupuncture operation will be affected if the pressure is too small; and if the pressure value is between the minimum threshold value and the maximum threshold value, the vibration device 5 enters the vibration working mode to realize the corresponding vibration modes and the acupuncture modes.

Particularly, the acquisition device is used for acquiring the vibration parameters of external shallow-needle acupuncture techniques and transmitting the same to the microprocessor in the control device. The microcontroller receives the electrical signals of the vibration parameters, processes the waveform coding and decoding, obtains the waveform data of vibration modes corresponding to that selected with the button, and outputs the data to the power amplifier circuit. The electrical signals of the waveform data of vibration modes are amplified by the power amplifier circuit and transmitted to the vibration generation component. The amplified electric signals drive the vibration generation component 51 to vibrate. The generated vibration is then transmitted to the vibrating head 50, thereby reproducing the vibration waves of shallow-needle acupuncture techniques. If the pressure value is greater than the maximum threshold value, the vibration device 5 does not enter vibration working mode because such pressure will cause discomfort to the acupuncture recipient. Obviously, it is also possible to set only the minimum threshold value without setting the maximum threshold value; when the pressure is too high, the acupuncture recipient usually consciously relieves the discomfort by adjusting the tightness of the strap. Apparently, the minimum threshold value and the maximum threshold value may be selected and set according to actual needs, and no specific restrictions are made here. In accordance with embodiments of the invention, an embodiment of a method for simulating the features of shallow-needle acupuncture techniques is provided. It should be noted that the steps illustrated in the flowchart drawings may be performed in a computer system which can execute a set of computer-executable instructions, and although a logical sequence is illustrated in the flowchart, the steps illustrated or described may be performed in an order different from that described herein in some instances.

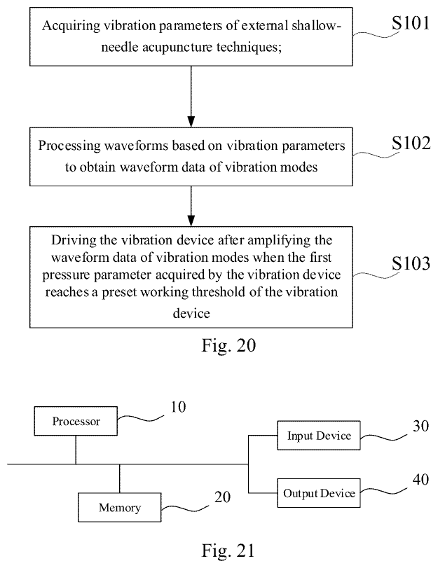

The embodiment provides a method for simulating the features of shallow-needle acupuncture techniques, which may be used in the above-described system for simulating the features of shallow-needle acupuncture techniques. FIG. 20 is the flowchart of the method for simulating the features of shallow-needle acupuncture techniques according to the embodiment of the invention. As shown in FIG. 20, the flowchart includes the following steps:
Step S101: acquiring the vibration parameters of external shallow-needle acupuncture techniques.

Particularly, the acquired vibration parameters of the external shallow-needle acupuncture techniques are the vibration parameters of the external shallow-needle acupuncture techniques which are obtained by the acquisition device 2. As shown in FIG. 4, the acquisition device 2 includes a vibration signal acquisition module 21 and a data processing module 23, wherein the vibration signal acquisition module 21 is connected to the data processing module 23, and the data processing module 23 is connected to the control device.

The vibration signal acquisition module 21 is used to acquire vibration signals of external shallow-needle acupuncture techniques and transmit the vibration signals to the data processing module 23, wherein the vibration signals are acceleration vibration signals along the direction of three axes. Particularly, the acceleration vibration signals along the direction of three axes are acceleration vibration signals on X-axis, Y-axis and Z-axis. As shown in FIG. 5, the vibration signal acquisition module 21 is used to acquire the vibration signals generated when the helix coil 41 on the shallow needle 4 is scraped (the shallow needle 4 is a manually operated acupuncture needle by itself alone). The data processing module 23 is electrically connected to the vibration signal acquisition module 21, and receives, analyzes and processes the vibration signals to obtain vibration parameters.

As shown in FIG. 6, the vibration signal acquisition module 21 includes an acceleration sensor 211 and a wire 212. As shown in FIG. 7, the acceleration sensor 211 is bonded to one side of the shallow needle 4 with glue so that the acceleration sensor 211 is perpendicular to the lengthwise direction of the shallow needle 4. This setup makes the vibration by scraping shallow needle 4 become most significant along the Z-axis direction of the acceleration sensor 211. One end of the wire 212 is connected to the acceleration sensor 211, and the other end is connected to the data processing module 23 via the USB interface. The data processing module 23 is a computer. The acceleration sensor 211 is a mature commercial product, and its structure will not be elaborated here.

As shown in FIG. 7, the shallow needle 4 is fixed on the side of the acceleration sensor 211 during acquisition. Scraping the helix coil 41 on the shallow needle 4 makes the shallow needle 4 vibrate. Using the acceleration sensor with supporting software to acquire the three-axe vibration acceleration values. The data processing module 23 performs data screening and data denoising on the vibration signals, obtains vibration parameters, and transmits the parameters to the control device 1.

The acquisition device further includes a pressing-pressure signal acquisition module 22 which is used to acquire pressing-pressure signals of external shallow-needle acupuncture techniques and transmit the pressing-pressure signals to the data processing module 23; the data processing module 23 is electrically connected with the pressing-pressure signal acquisition module 22, receives, analyzes and processes pressing-pressure signals, and obtains the second pressure parameter.

Particularly, the pressing-pressure signal acquisition module 222 includes a push-pull force gauge and a wire. The push-pull force gauge is connected to the data processing module 23 via the wire. During acquisition, the acupuncturist presses the bottom of the shallow needle 4, so that the tip of the shallow needle 4 is vertically pressed against the push-pull force gauge which acquires 1,700 pressing-pressure data for about 170 seconds, and generates 1,700 pressing-pressure signals. These signals are transmitted to the data processing module 23 through the wire. The data processing module 23 performs data screening and data denoising on the pressure signals, obtains the second pressure parameters and transmits the parameters to the control device 1.
Step S102: processing waveforms based on the acquired vibration parameters to obtain waveform data of vibration modes.

Particularly, the control device 1 is used to store the vibration parameters of shallow-needle acupuncture techniques, process waveforms based on the vibration parameters, and obtain the waveform data of vibration modes. When the first pressure parameter obtained by the vibration device reaches the preset working threshold of the vibration device, the control device drives the vibration device to vibrate after amplifying the waveform data of vibration modes. The control device 1 in this step processes the second pressure parameter. The pressing-pressure waveform data are obtained to provide a reference for pressing-pressure threshold value to the vibration device for vibration.

Particularly, the vibration parameters are stored in the storage module of the control device via the USB interface, and the vibration parameters are presented in the form of vibration time-domain waveforms and vibration spectrum after waveforms are processed by the microcontroller. The generated vibration time-domain waveforms include the original vibration time-domain waveforms of acupuncture techniques and the synthesized vibration time-domain waveforms of simulating acupuncture techniques, so that the vibration device 5 has two vibration modes, which can be selected specifically by a set of buttons on the control device. The original vibration time-domain waveform of acupuncture techniques is the PCM waveform sequence data that replicates the acupuncture techniques of acupuncturists.

FIGS. 8, 9, and 10 are typical vibration time-domain waveforms corresponding to X, Y, and Z axes, respectively. As shown in these figures, Wave A in the first amplitude range (shown in FIG. 10) and Wave B in the second amplitude range (shown in FIG. 10) alternatively present on all the three axes (namely X, Y, and Z axes), with the most significant effect on the Z axis. The adjacent first-amplitude wave and second-amplitude wave, correspond to one complete upward and downward scraping operation of the shallow needle 4 for reinforcing or reducing method. The Z-axis is the direction perpendicular to the human body surface. The vibration along Z-axis is the major direction of upward and downward vibration generated by upward and downward scraping of shallow needle coil, and also the major physical energy that the shallow needle acts on the human body. The X-axis and Y-axis are directions parallel to the human body surface, and they are additional directions of vibration generated by upward and downward scraping of shallow needle coil. The vibration along X-axis and Y-axis is not the major physical energy acting on the human body. FIGS. 8, 9, and 10, are typical vibration time-domain waveforms corresponding to X, Y, and Z axes respectively. The vibration amplitude on Z-axis is significantly greater than that on X-axis and Y-axis. The first and the second amplitude waves on Z-axis clearly correspond to the light and heavy features of shallow needle scraping techniques, while the first and second amplitude waves on X-axis and Y-axis do not clearly correspond to the light and heavy features of the shallow needle scraping techniques. Therefore, the vibration waveform implemented by the shallow acupuncture instrument select the Z-axis waveform data, and generate vibration on the Z-axis direction.

Particularly, both the original vibration time-domain waveforms of acupuncture techniques and the synthesized vibration time-domain waveforms of simulating acupuncture techniques include: the waveform of the reinforcing acupuncture method, the waveform of the reducing acupuncture method, and the waveform of the even reinforcing-reducing acupuncture method. As shown in FIG. 10, the waveform of the reinforcing acupuncture method corresponds to the acupuncture technique of lightly upward scraping and heavily downward scraping, featuring the vibration waveform amplitude of upward scraping in the first half (as Wave A in the first amplitude range shown in FIG. 10) is lower than that of the downward scraping in the second half (as Wave B in the second amplitude range shown in FIG. 10). The waveform of the reducing acupuncture method corresponds to the acupuncture technique of heavily upward scraping and lightly downward scraping, featuring the vibration waveform amplitude of upward scraping in the first half (as Wave B in the first amplitude range shown in FIG. 10) is greater than that of the downward scraping in the second half (as Wave A in the second amplitude range shown in FIG. 10). The waveform of even reinforcing-reducing acupuncture method, corresponds to the acupuncture technique of evenly upward and downward scraping, featuring the vibration waveform amplitude of upward scraping in the first half basically equals to that of downward scraping in the second half. The waveforms of the reinforcing acupuncture method, the reducing acupuncture method and the even reinforcing-reducing acupuncture method enable the vibration device 5 to have three modes of shallow acupuncture techniques (reinforcing, reducing and even reinforcing-reducing) to choose, specifically using another set of buttons on the control device.

When the control device performs waveform processing based on the second pressure parameter, the data processing module 23 selects 500 pressing-pressure signals at a stable period (with a duration of about 50 seconds, 45 fluctuation cycles, and an average cycle of 1.1 seconds) from 1,700 pressing-pressure signals (shown in FIG. 11). The corresponding pressing-pressure waveforms of the shallow-needle acupuncture technique after waveform processing are shown in FIG. 12, and the pressing-pressure signals are used to obtain the following results: the average pressing-pressure is 84 g, with the maximum value being 126 g, and the minimum value being 29 g. Referring to four expert doctors in the rehabilitation department of a rehabilitation hospital, each of them used three shallow-needle acupuncture techniques (reinforcing, reducing and even reinforcing-reducing, respectively), a total of 12 groups of pressing-pressure data are acquired, and the pressing pressure range is determined as 53 g to 152 g, in order to avoid that the pressing pressure is too small to affect the effect of shallow acupuncture operation. Apparently, the number of pressing-pressure parameters acquired by the push-pull force gauge can also be other values, and the number of selected pressing-pressure signals at a stable period can also be other values. It should be noted that the pressing pressure acquired by the push-pull force gauge is a pressing pressure formed by the superposition of three factors: the first factor is a relatively constant pressure generated by the acupuncturist by pressing on the bottom of the shallow needle 4; the second factor is a pressure fluctuation at about 1 Hz with the movement of the shallow needle 4; and the third factor is a vibration wave of about 200 Hz brought about by scraping the helix coil 41 on the shallow needle 4, i.e., the pressing-pressure is not constant, which means the pressure waveform data of the shallow-needle acupuncture technique includes pressure waveform data of both constant pressure and variable pressure.
Step S103: driving the vibration device to vibrate after amplifying the waveform data of vibration modes, when the first pressure parameter acquired by the vibration device reaches a preset working threshold of the vibration device. Particularly, the waveform data of vibration modes is obtained by the control device 1, and when the first pressure parameter obtained by the vibration device reaches the preset working threshold value of the vibration device, the power amplifier circuit 12 in the control device 1 amplifies the waveform data of vibration modes and drives the vibration generation component of the vibration device 5 to vibrate. The generated vibration is then transmitted to the vibrating head 50, thereby simulating the shallow-needle acupuncture technique applied on the acupuncture recipient.

The methods provided by the embodiment for simulating the features of the shallow-needle acupuncture techniques, which includes acquiring vibration parameters of external shallow-needle acupuncture techniques; processing waveforms based on the acquired vibration parameters to obtain waveform data of vibration modes; driving the vibration device to vibrate after amplifying the waveform data of vibration modes when the first pressure parameter acquired by the vibration device reaches the preset working threshold of the vibration device, realizes driving the vibration device of the shallow acupuncture instrument to vibrate according to the waveform data of vibration modes, generating vibration waves that can reproduce the shallow-needle acupuncture techniques, to address the difficulty in reproducing vibration waves of shallow-needle acupuncture techniques.

The embodiments of the invention also provide a computer device as shown in FIG. 21 which is a schematic diagram of the computer device provided by an optional embodiment of the invention. As shown in FIG. 21, the computer device includes: one or more processors 10, a memory 20, and interfaces for connecting the various components, including high-speed interfaces and low-speed interfaces. The components are communicatively connected to each other utilizing different buses and may be mounted on a common motherboard or otherwise mounted as desired. The processor may process instructions executed within the computer device, including instructions stored in or on memory to display graphical information of the GUI on an external input/output device (e.g., a display device coupled to the interface). In some optional implementations, multiple processors and/or multiple buses may be used with multiple memories and multiple memories, if desired. Similarly, a plurality of computer devices may be connected, with the individual devices provided with some of the necessary operations (e.g., as an array of servers, a group of blade servers, or a multiprocessor system). FIG. 21 is shown with a processor 10 as an example.

The processor 10 may be a central processor, a network processor, or a combination thereof. Among other things, the processor 10 may further include a hardware chip which may be a specialized integrated circuit, a programmable logic device, or a combination thereof. The programmable logic device may be a complex programmable logic device, a field programmable logic gate array, a general-purpose array logic or any combination thereof.

The memory 20 stores instructions executable by at least one of the processors 10, so that at least one of the processors 10 can execute the method illustrated in the above embodiment.

The memory 20 may include a storage program area and a storage data area, wherein the storage program area may store an operating system, an application program required for at least one function, and the storage data area may store data created based on the use of the computer device. In addition, the memory 20 may include a high-speed random access memory, and may also include a non-transient memory, such as at least one disk memory, a flash memory, or other non-transient solid state memory. In some optional implementations, the memory 20 may optionally include memories remotely located relative to the processor 10. These remote memories may be connected to the computer device via a network. Examples of the networks include, but are not limited to, the Internet, an enterprise intranet, a local area network, a mobile communications network, and a combination thereof.

The memory 20 may include volatile memory, e.g., a random access memory; the memory may also include non-volatile memory, e.g., flash memory, a hard disk, or a solid state drive; and the memory 20 may also include a combination of the above memories.

The computer device further includes an input device 30 and an output device 40. The processor 10, the memory 20, the input device 30, and the output device 40 may be connected via a bus or otherwise. FIG. 21 is shown with the connection via a bus.

The input device 30 may receive incoming numeric or character information, as well as generate inputs of key signals related to user settings as well as the function control of the computer device, such as a touch screen, a keypad, a mouse, a trackpad, a touchpad, an indicator bar, one or more mouse buttons, a trackball, a joystick, etc. The output device 40 may include a display device, an auxiliary lighting device (e.g., an LED), and a haptic feedback device (e.g., a vibration motor), etc. The aforementioned display devices include, but are not limited to, liquid crystal displays, light emitting diodes, monitors, and plasma displays. In some optional embodiments, the display device may be a touch screen.

The embodiment of the invention also provides a computer-readable storage medium, wherein the above-described method according to the embodiment of the invention may be implemented in hardware, firmware, or implemented as computer codes that may be recorded on a storage medium, or as computer codes originally stored on a remote storage medium or non-transitory machine-readable storage medium downloaded from a network, and as computer codes that will be stored on a local storage medium, so that the method described herein may be processed by such software stored on a storage medium using a general purpose computer, a dedicated processor, or programmable or specialized hardware. The storage medium may be a magnetic disk, a CD-ROM, a read-only storage memory, a random storage memory, a flash memory, a hard disk, or a solid state drive; further, the storage medium may include a combination of the above types of memories. It is understandable that the computer, processor, microprocessor controller, or programmable hardware includes a storage component that can store or receive software or computer codes, and when the software or computer code is accessed by the computer, processor, or hardware and is executed, the method illustrated in the embodiment is realized.

While the embodiments of the invention have been illustrated and described with drawings, many modifications and variations that can be made by those of ordinary skill in the art without departing from the spirit and scope of the invention, fall into/within the protection scope of the attached claims.

1. A control device of a shallow acupuncture instrument, wherein the shallow acupuncture instrument further includes a vibration device, the control device includes a microcontroller and a power amplifier circuit, wherein
the microcontroller is connected with the power amplifier circuit;
the microcontroller is used to process waveforms based on vibration parameters acquired from external inputs, obtain the waveform data of vibration modes, and transmit the obtained waveform data to the power amplifier circuit; the waveform data of vibration modes include: original vibration time-domain waveforms of shallow-needle acupuncture techniques, and e synthesized vibration time-domain waveforms of simulating shallow-needle acupuncture techniques,
wherein the original vibration time-domain waveform data of acupuncture techniques are the waveform data of the shallow-needle acupuncture techniques of TCM practitioners which are time-domain waveform amplitude data obtained through sampling procedures performed at a fixed sampling rate, the microcontroller reads the waveform amplitude data, and generates PCM waveform sequence data that replicate the shallow-needle acupuncture techniques of TCM practitioners,
wherein the synthesized vibration time-domain waveform data of simulating acupuncture techniques is generated through low-frequency modulation of amplitude of intermediate-frequency wave, the frequency of intermediate-frequency sinusoidal wave is a first preset threshold value, or the frequency of the low-frequency half-sinusoidal wave is a second preset threshold value, the configuration script data of the synthesized waveforms in the control device of the shallow acupuncture instrument is used to generate the PCM waveform sequence data of simulating shallow-needle acupuncture techniques;
when the first pressure parameter acquired by the vibration device reaches the preset working threshold, the power amplifier circuit drives the vibration device to vibrate after amplifying the waveform data of vibration modes;
the vibration parameters acquired from external inputs are obtained in the following way:
acquiring vibration signals of external shallow-needle acupuncture techniques, analyzing and processing the vibration signals to obtain the vibration parameters, wherein the vibration signals are acceleration vibration signals along the direction of three axes; and
the microcontroller is used to collect a second pressure parameter of external shallow-needle acupuncture techniques, process waveforms based on the second pressure parameter, obtain the pressing-pressure waveform data, and provide a pressing-pressure threshold value for the vibration device of the shallow acupuncture instrument.

2. The control device according to claim 1, wherein the control device further includes a pressure detection circuit that is connected to the microcontroller; the pressure detection circuit is used for calculating the pressure value of the first pressure parameter acquired by the vibration device and transmitting the pressure value to the microcontroller; and the microcontroller controls the startup and shutdown of the vibration device according to the relationship between the pressure value and the preset working threshold value of the vibration device.

3. The control device according to claim 1, wherein both the original vibration time-domain waveforms of shallow-needle acupuncture techniques and the synthesized vibration time-domain waveforms of simulating shallow-needle acupuncture techniques include the reinforcing acupuncture waveform data, the reducing acupuncture waveform data, and the even reinforcing-reducing acupuncture waveform data, which correspond to waveforms of the reinforcing acupuncture method, reducing acupuncture method, and even reinforcing-reducing acupuncture method, respectively.

4. The control device according to claim 1, wherein the control device further includes a storage module, a USB interface, an indicator light, and a button which are all connected to the microcontroller;
the USB interface is used to import externally inputted vibration parameters and second pressure parameters into the storage module, and to provide a charging interface for the control device;
the storage module is used for receiving the vibration parameters and the second pressure parameters imported via the USB interface and transmitting the vibration parameters and the second pressure parameters into the microcontroller;
the indicator light is used for displaying the working status of the shallow acupuncture instrument; and
the button is used for selecting the vibration mode of the vibration device.

5. The control device according to claim 1, wherein the control device further includes a wireless communication module which is connected to the microcontroller and used to provide wireless communication signals to the control device.

6. A shallow acupuncture instrument, comprising a vibration device and a control device as claimed in claim 1, wherein
the control device is electrically connected to the vibration device;
the control device is used to process waveforms based on vibration parameters acquired
from external inputs, and obtain the waveform data of vibration modes; when the first pressure parameter acquired by the vibration device reaches the preset working threshold of the vibration device, the control device drives the vibration device to vibrate after amplifying the waveform data of vibration modes.

7. The shallow acupuncture instrument according to claim 6, wherein the vibrating device of the shallow acupuncture instrument includes a vibrating head and a vibration generation component; the first end of the vibrating head is connected to the vibration generation component, and the second end of the vibrating head is contacting acupuncture recipient;
the vibration generation component is used to generate vibration according to the waveform data of vibration modes which is amplified by the power amplifier circuit of the control device, to drive the vibrating head to vibrate.

8. The shallow acupuncture instrument according to claim 7, wherein the vibration device further includes a pressure sensor which is connected to the pressure detection circuit of the control device; the pressure sensor is used for obtaining the first pressure parameter applied by the vibrating head to acupuncture recipient when the vibration device is not in operation, and then transmitting the first pressure parameter to the pressure detection circuit of the control device; the pressure detection circuit is used to calculate the pressure value of the first pressure parameter applied by the vibrating head to acupuncture recipient when the vibration device is not in operation, and then transmit the pressure value to the microcontroller of the control device; and the microcontroller controls the startup and shutdown of the vibration device according to the relationship between the pressure value and the preset working threshold value of the vibration device.

9. The shallow acupuncture instrument according to claim 6, wherein the control device and the vibration device are of integrated design.

10. A system for simulating the features of shallow-needle acupuncture techniques, wherein the system includes an acquisition device and the shallow acupuncture instrument according to claim 6; the acquisition device is electrically connected to the control device;
the acquisition device is used for acquiring vibration parameters of shallow-needle acupuncture techniques;
the control device is used to process waveforms based on vibration parameters, and obtain the waveform data of vibration modes; when the first pressure parameter acquired by the vibration device reaches the preset working threshold of the vibration device, the control device drives the vibration device to vibrate after amplifying the waveform data of vibration modes.

11. The system according to claim 10, wherein the acquisition device is used for acquiring the second pressure parameter of external shallow-needle acupuncture techniques and transmitting the second pressure parameter to the control device.

12. The system according to claim 11, wherein the acquisition device includes a vibration signal acquisition module, a pressing-pressure signal acquisition module and a data processing module; both the vibration signal acquisition module and the pressing-pressure signal acquisition module are connected to the data processing module; and the results of the data processing module are transmitted to the control device;
the vibration signal acquisition module is used to acquire vibration signals of external shallow-needle acupuncture techniques and transmit the vibration signals which are three-axis acceleration vibration signals to the data processing module;
the pressing-pressure signal acquisition module is used to acquire pressure signals of external shallow-needle acupuncture techniques and transmit the pressing-pressure signals to the data processing module;
the data processing module receives vibration signals and pressing-pressure signals, and analyzes and processes vibration signals and pressure signals, respectively, to obtain the vibration parameter and the second pressure parameter, and to transmit the vibration parameter and the second pressure parameter to the control device;
the control device is further used to process waveforms based on the second pressure parameter, obtain the pressing-pressure waveform data, and provide a pressing-pressure threshold value for the vibration device during vibration.

13. A method for simulating the features of shallow-needle acupuncture techniques, wherein the method is applied to the system for simulating the features of shallow-needle acupuncture techniques according to claim 10; the method includes:
acquiring vibration parameters of external shallow-needle acupuncture techniques;
processing waveforms based on the acquired vibration parameters to obtain waveform data of vibration modes;
driving the vibration device to vibrate after amplifying the waveform data of vibration modes when the first pressure parameter acquired by the vibration device reaches the preset working threshold of the vibration device.

14. A computer device, comprising a memory and a processor which are communicatively connected, wherein computer instructions are stored in the memory, and the processor executes the method of simulating the features of shallow-needle acupuncture techniques according to claim 13 by executing the computer instructions.

15. A computer-readable storage medium, wherein computer instructions are stored on the computer-readable storage medium and used to enable the computer to execute the method of simulating the features of shallow-needle acupuncture techniques according to claim 13.
